(19) United States
(12) Patent Application Publication
(10) Pub. No.: US 2010/0167277 A1
(43) Pub. Date: Jul. 1, 2010

(54) Fret sequencing by DNA scanning proteins

(76) Inventor: Ryan Anthony Harper, Berkeley, CA (US)

Correspondence Address: RYAN HARPER, 1423 MLK #A5, BERKELEY, CA, 94709, US

(21) Appl. No.: 12/317,680

(22) Filed: Dec. 29, 2008

(51) Int. Cl. C12Q 1/68

(52) U.S. Cl. 435/6

(57) ABSTRACT

This patent describes a novel method for DNA sequencing. A DNA hybrid is created such that one strand contains nucleotides (A, G, C, T/U) bound to different FRET acceptor fluorophores that emits a distinct, differentiable wavelength. A DNA scanning protein carrying a FRET donor fluorophore is also added. A laser with a wavelength that excites the donor molecule irradiates the reaction. As the protein complex passes over a nucleotide on the DNA strand, a FRET reaction occurs between the donor and acceptor fluorophores. The acceptor fluorophore will emit a distinct wavelength which is detected and correlated to a specific nucleotide. As the DNA scanning protein moves along the DNA molecule the entire sequence of nucleotides can be determined by correlating the wavelengths emitted with the specific nucleotide associated with it.

### BACKGROUND

The Sanger dideoxy chain termination DNA sequencing method was the most popular sequencing method of the late 20^{th} century. In this method, dideoxynucleotides (ddAPT, ddCTP, ddGTP and ddTTP) were used to terminate a growing DNA chain because they do not contain a 3′ OH group which is necessary for the DNA polymerization. Four separate sequencing reactions are carried out, all containing the same reagents—DNA polymerase, dNTPs, primer, and template DNA. However each tube receives a different ddNTP. Because the polymerase has a choice to choose between a normal dNTP and a chain terminating ddNTP, the newly synthesized DNA will vary in lengths. The four tubes, (ddATP tube, ddCTP tube, ddGTP tube and the ddTTP tube) are electrophoresed in separate wells of a polyacrylamide gel. The sequence of the DNA strand can be determined by analyzing the bands in each of the four wells (Sanger et al Proc. Natl. Acad. Sci. USA 74:5463 which is incorporated herein by reference)

More modern automated DNA sequencing machines use fluorescently labeled nucleotides which are read by a detector. However, the DNA still needs to be electrophoresed. The electrophoresis step limits this sequencing method because the number of nucleotides that can be determined in a single sequencing run is less than 1000 base pairs. This limitation has lead many attempts to sequence DNA on a molecular level—using only one piece of DNA rather than the large number needed for gel electrophoresis.

Jett and Ulmar (U.S. Pat. No. 4,962,027 and U.S. Pat. No. 5,674,743) developed a molecular sequencing method in which they synthesize a complementary strand of DNA using fluorescently labeled nucleotides. Once that strand was synthesized an exonuclease is added to cleave the fluorescently labeled nucleotides on the DNA molecule. The nucleotides flow through a detector and specific nucleotides identified. However, the DNA molecule must be held in a stream which causes the molecule to shear.

The emergence of high throughput sequencing machines, such as those developed by 454 life science as seen some commercial success. In the 454 method developed by Rothberg (Nature 2006 May 4:441(7089):120 which is incorporated herein by reference), many small single DNA fragments are bound to beads in separate wells. A specific nucleotide is washed over the wells one at a time. If a light reaction occurs in the well, a specific nucleotide is incorporated into the growing chain. However, only small lengths of DNA can sequenced. In addition, 454 Life Science recently predicted that it would take two years to sequence a Neanderthal genome. Much too long and expensive for sequencing individuals in clinical applications.

The use of FRET (Fluorescence Resonance Energy Transfer) DNA Sequencing was first implemented by Ju and Mathies (U.S. Pat. No. 5,814,454 and U.S. Pat. No. 5,707,804). FRET occurs when a donor fluorophore is excited to a higher energy state by a specific wavelength of light. The donor molecule can then transfer energy to an appropriate acceptor molecule if they are in close physical proximity and the emission and absorbance spectra overlap. However, the method described by Ju and Mathies still required an electrophoresis step.

Schneider and Rubens (U.S. Pat. No. 6,982,146) invented a system in which DNA was sequenced using unique fluorescent FRET acceptor molecules bound to each of the four nucleotides. A FRET donor molecule is attached to a DNA polymerase. As the polymerase places the nucleotide onto the growing DNA chain, a laser excites the donor molecule which transfers it's energy to the acceptor fluorophore on the nucleotides. However, free unbound nucleotides could cause high interference and complicate the detection process. In addition there is reason to believe that as the DNA polymerase adds the fluorescent nucleotides, the polymerase chops off the fluorescent tag making the method unreliable (B. A. Mulder et al., Nucleic Acids Res. 2005 33, 4865, U.S. Pat. No. 6,399,335 and S. Kumar et al., Nucleos. Nucleot. Nucleic Acids 2005 24, 401 which are incorporated herein by reference). Thus, there still exist a need to sequence DNA (or RNA) on a molecular level in a fast efficient manner.

The FRET DNA sequencing approach taken by many scientist has several problems. As just mentioned, the DNA polymerase tends to cut off the labeled molecule as it adds the nucleotide to the growing chain. Recently John Eid et al. (Science Nov. 20, 2008.—epub ahead of print which is incorporated herein by reference) used this problem to their advantage by bonding a DNA polyemerase to a solid surface and and as it adds the flourescently labeled nucleotide to the DNA chain they detect flourescent molecule as DNA polmerase chops it off.

The method described here provides an alternative method for identifying nucleotides on a single nucleic acid molecule.

### SUMMARY OF PATENT

This patent describes a method for sequencing DNA on the molecular level that uses FRET as a detection system. The method allows long DNA (or RNA) molecules to be sequenced quickly. The method also allows many different reactions to occur on the same substrate and recorded by the same piece of equipment.

A template DNA (or RNA) molecule to be sequenced is fluorescently labeled on one strand such that each nucleotide (A, G, C, T/U) contains a different fluorophore that emits a distinct and differentiable wavelength. A DNA scanning protein containing a FRET donor fluorophore is added the nucleic acid molecule. As the FRET donor fluorophore labeled protein scans the DNA chain, it passes over fluorescently labeled nucleotides on the fluorescent strand. A laser excites the FRET donor molecule on the DNA scanning protein, but not the acceptor fluorophores on the fluorescent DNA strand. For FRET to work, donor fluorophore and the acceptor fluorophore must be in close proximity and the donor must be excited by a wavelength of light that does not excite the acceptor fluorophores. In addition, the donor fluorophore must emit light that falls within the acceptor's absorbance spectrum.

As the DNA scanning protein passes over the labeled nucleotides on the fluorescent strand, a FRET reaction occurs between the excited donor fluorophore on the protein and the acceptor molecule on the nucleotides. The acceptor fluorophore emits light at a specific wavelength which is detected by a detector. Because different nucleotides (A, T/U, G, C) are labeled with different and distinguishable fluorophores, the detection of a specific wavelength can be correlated to a specific nucleotide.

The is a vast improvement over the FRET sequencing methods described in the background. This method does not require an electrophoresis step and it does not necessarily require the DNA scanning protein to be a DNA polymerase, although in some embodiments described below it can be. The use of a non-polymerase as a DNA scanning protein is important. Other methods such as the one described by Schneider and Rubens (U.S. Pat. No. 6,982,146) use a DNA-polymerase-fluorophore fusion protein to add flourescent nucleotides to a growing DNA chain. FRET activity could be detected between this DNA polymerase fusion protein and the flourescent nucleotides it is adding. However, other scientist have shown that the fluorescent tag on the nucleotides gets chopped off as the polymerase adds them to the growing strand, making these types of strategies ineffective (B. A. Mulder et al., Nucleic Acids Res. 2005 33, 4865, U.S. Pat. No. 6,399,335 and S. Kumar et al., Nucleos. Nucleot. Nucleic Acids 2005 24, 401 which are incorporated herein by reference). In addition, by removing unbound fluorescent nucleotides from the FRET reaction, the method described in this application will have less background signal than the method described by Schneider and Rubens (U.S. Pat. No. 6,982,146).

In a different embodiment, a luminescent protein can be used on the DNA polymerase in place of the FRET donor fluorophore. In this case, a laser is not needed to irradiate the reaction.

In another embodiment of this method, a RNA molecule can be sequenced. RNA is converted to single stranded cDNA. The cDNA can be used as the template strand in the DNA replication reaction and will be treated the same as described above.

In particular embodiments, it many be advantageous to use multiple donor fluorophores attached to the DNA scanning protein complex. FRET reactions required that the donor fluorophore emits light at a wavelength that is within the acceptor's absorbance spectrum. A combination of acceptor fluorophores may require a wider excitation spectrum than what is possible by a single donor fluorophore. In this case, two or more different lasers with different wavelengths may be required to excite the donor molecules on the protein.

Another embodiment exist in which a DNA scanning protein complex, such as that used in mismatch repair, is used instead of a single DNA scanning protein.

An important embodiment of this method is the use of DNA polymerase as the scanning protein. In this case, the fluorescently labeled single stranded nucleic acid molecule is incubated with DNA Polymerase bound to the donor FRET fluorophore. A primer, unlabeled nucleotides and the appropriate cofactors and buffers are also added. As the DNA polymerase builds the new DNA strand, it passes over the labeled nucleotides the donor fluorophore is excited with a laser and a FRET reaction occurs between the donor fluorophore on the polymerase and the acceptor fluorophore on the template strand. Although this embodiment does use a DNA polymerase as the DNA scanning protein, it is an improvement over the Schneider and Rubens (U.S. Pat. No. 6,982,146) method because the DNA polymerase-fluorophore fusion protein is not adding flourescent nucleotides, it adds normal unlabeled nucleotides, but passes over the fluorescent nucleotides on the template strand. As mentioned previously, in some cases the DNA polymerase has been shown to eliminate the fluorescent molecule on the nucleotide as it extends the growing DNA chain making the Schneider and Rubens (U.S. Pat. No. 6,982,146) system inefficient and unreliable. This new method circumvents this problem by labeling the template strand, possibly by chemical or other means, so that a FRET reaction can occur between DNA polymerase-fluorophore fusion protein and the labeled template strand as the polymerase adds unlabeled nucleotides.

Many different embodiments exist for creation of flourescently labeled DNA and the detection of the excited acceptor fluorophore. Some of these methods are described in detail in Patent number U.S. Pat. No. 6,982,146 B1 and U.S. Pat. No. 594,531.

### Detailed Embodiment 1—PCNA as a DNA Scanning Protein

The following is a novel method for sequencing nucleic acids. The method relies on the incorporation of fluorescent nucleotides in one strand of a double stranded DNA molecule. A distinct and distinguishable FRET acceptor fluorophores are placed on each type of nucleotide (FIG. 1). This can be done enzymatically using DNA polymerase or by chemical means. Gerald Giller et al is one of many that describe in detail the production of a flourescently labeled DNA strand (Nucleic Acid Research 2003 May 15: 31(10):2630-2635 which is incorporated herein by reference).

Many embodiments exist for this first DNA replication reaction. For example, the initial template DNA molecule might be physically or chemically bound to a substrate. Alternatively, the primer used in the reaction could be physically or chemically bound to a substrate. This substrate could be a glass slide, well, or a bead.

Once the nucleotide labeling reaction is complete and a labeled/unlabeled DNA hybrid is created, the labeled/unlabeled nucleic acid polymer is purified. Again, this can be accomplished in many ways depending on how the initial labeling reaction was done. One possible embodiment is that the DNA is bound (by the primer or template DNA) to a substrate such as a glass slide and the reaction is washed removing the undesired reagents from the reaction. Binding DNA to solid substrates are discussed by Zhao (Zhao et al. Nucleic Acids Res. Feb. 15, 2001; 29(4): 955-959 which is incorporated herein by reference) Adessi (Adessi et al. Nucleic Acids Res. Oct. 15, 2000;28(20):E87 which is incorporated herein by reference) and Matsuura (Matsuura et al. J Biomol Struct Dyn. Dec. 20, 2002;20(3):429-36 which is incorporated herein by reference). Alternatively the nucleic acid molecule can be purified using one of the many commercially available chromatography columns and is easily accomplished by those skilled in the art.

Once the labeled/unlabeled DNA hybrid is purified the DNA scanning reaction can begin. The appropriate buffer and a FRET donor fluorophore bound to a DNA scanning protein are added to the reaction. All reagents to allow DNA scanning to begin must also be added. In one embodiment, PCNA, a trimer that creates a circular ring that moves along the DNA molecules, is bound to GFP (absors 488 nm and emits at 510 nm). This can be accomplished by using recombinant DNA technology and those skilled in the art are aware of how to generate and purify a PCNA-GFP fusion proteins.

The PCNA-GFP fusion protein can be bound to a substrate, or alternatively the flourescently labeled DNA hybrid could be bound, by one or both ends, to a substrate. When mixed with the appropriate co-factors, buffer and other potentially necessary proteins, such a RFC which loads PCNA onto the DNA, the DNA scanning complex binds to the DNA and will move along it. PCNA-GFP passes over the FRET acceptor fluorophores attached to the nucleotides that make up fluorescent strand (see FIG. 2). A FRET donor/acceptor combination must be chosen that allows a FRET reaction to be possible—meaning that the donor fluorophore emits light at a wavelength that can excite the acceptor molecule. The following are examples of possible candidates for FRET acceptor fluorophores, Cl Nerf (absorbs at 514 nm and emits at 540 nm), Calcium green-1 Ca²⁺ Dye (absorbs at 509 nm and emits at 530 nm), Organ Green (absorbs at 506 nm and emits at 526 nm), and FM—4-46 (absorbs at 515 nm and emits at 640 nm). These examples were chosen purely on there absorbance spectrum, their ability to be conjugated to specific nucleotides, their efficiency in the FRET reaction and their ability to be incorporated into DNA will need to be investigated. Nucleotides bound to fluorophores can also be purchased commercially. Various techniques have also been developed to attach fluorophores to nucleotides. These techniques can be found in U.S. Pat. No. 5,047,519, Corrie et al. 1999, Nature 400:425, and U.S. Pat. No. 5,151,507 .

As the PCNA-GFP fusion protein moves past the fluorescently labeled nucleotides, a laser excites the donor fluorophore on the DNA scanning protein causing a FRET reaction between the donor fluorophore and a nearby acceptor fluorophore. The wavelength used to excite the donor fluorophore must not excite any of the acceptor fluorophores. In this case using GFP, a 488 nm laser is used. Those skilled in the art would be able to assign other lasers to specific donor fluorophores.

In some embodiements that use a Mismatch Repair Complex as the DNA scanning complex, it might be advantageous to find a mutant protein complex that can pass over the fluorescently labeled nucleotides without stopping to remove the nucleotide.

In some embodiments, it may be possible to chemically bind the the donor fluorophore directly to the PCNA molecule instead of creating a PCNA-GFP fusion protein. Description of how to bind some fluorescent dyes to proteins or antibodies are described by Verveer et al. (CSH Protocols; 2006; doi:10.1101/pdb.prot4645 which is incorporated herein by reference) and Tadatsu et al (The Journal of Medical Investigation 2006 February;53(1-2):52-60 which is incorporated herein by reference). In addition, many fluorescently labeled antibodies are available commercially.

Many different donor fluorophore DNA scanning complexes (or proteins) may have to be made and screened to find an optimal configuration. For example, you may find that for a particular combination of donor fluorophores and DNA scanning proteins that the donor fluorophore provides a better FRET reaction when fused on the C terminus instead of the N terminus of a particular protein. An example of a potential screen for effective fusion complexes would be generate a nicked plasmid that contains one strand that is fluorescently labeled. As many potential donor fluorophore/DNA scanning complexes as possible could be screened on glass slides by mixing the oligonucleotide substrate with the fusion protein and then exciting the donor with a laser. The FRET reaction produced by the donor and acceptor molecules can be detected in many different ways. U.S. Pat. No. 6,982,146 explains some different embodiments of how the FRET reaction can be detected. Once detected the emissions spectra from the acceptor molecules can be correlated to specific nucleotides.

There are many different clinical applications of this patent. Potentially, entire genomes could be sequenced within days at a relatively inexpensive price. It will offer individuals the potential to sequence their own genomes opening a new frontier into medicine, disease prevention, treatment and research.

Because of the potential for many different embodiments of this novel DNA sequencing method, it should be noted that the illustrated embodiments are only example and should not limit the scope of this patent.

### Detailed Embodiment 2—DNA Polymerase as a DNA Scanning Protein

The following is a novel method for sequencing nucleic acids using DNA Polymerase as a DNA scanning protein. The method relies on the incorporation of fluorescent nucleotides in one strand of a double stranded DNA molecule. A distinct and distinguishable FRET acceptor fluorophores are placed on each type of nucleotide. For example, dATP might be bound to BODIPY while dCTP might be bound to rhodamine and so on. Many fluorescent nucleotides are available commercially. DNA Polymerase will copy the template strand, incorporating the labeled nucleotides into the newly synthesized strand (see FIG. 1). This is done in a typical primer extension (DNA replication) reaction that can be accomplished by someone skilled in the art and is also described by in detail by Gerald Giller et al (Nucleic Acid Research May 15, 2003: 31(10):2630-2635 which is incorporated herein by reference). The end product of the reaction is a double stranded DNA molecule that has one normal strand and one strand that is fluorescently labeled.

Many embodiments exist for this first DNA replication and labeling reaction. For example, the initial template DNA molecule might be physically or chemically bound to a substrate. Alternatively, the primer used in the reaction could be physically or chemically bound to a substrate. This substrate could be a glass slide or a bead. In some embodiments, the template DNA is single stranded, but double stranded DNA could also be used. If double stranded DNA is used, the template DNA needs to be denatured so that the primer can bind to its target sequence.

Once the initial DNA replication and labeling reaction is complete and a labeled/unlabeled DNA hybrid is created, the unbound “free” fluorescently labeled nucleotides and the DNA polymerase needs to be removed from the reaction. In addition, the labeled/unlabeled DNA hybrid needs to become denatured so that the primer in the second DNA replication reaction can bind the target sequence on the fluorescent strand. Again, this can be accomplished in many ways. In some embodiments, the original primer used in the first synthesis reaction is bound to a substrate such as a glass slide and the reaction is heated to 96 degrees Celsius and if the labeled strand is anchored to a substrate, then undesired reagents (DNA polymerase and fluorescent dNTPs) can simply be washed away. Binding DNA to solid substrates are discussed by Zhao et al (Nucleic Acids Res. Feb. 15, 2001; 29(4): 955-959 which is incorporated herein by reference), Adessi et al (Nucleic Acids Res. Oct. 15,2000;28(20):E87 which is incorporated herein by reference) and Matsuura et al (J Biomol Struct Dyn. Dec. 20, 2002;(3):429-36 which is incorporated herein by reference). If the original template DNA was bound to the substrate, the reaction will need to be washed first to remove the DNA polymerase and fluorescent dNTPs and then heated to allow the labeled and unlabeled DNA molecules to separate. The labeled strand can then be collected by rinsing the reaction and removing the liquid which should contain the label nucleic acid molecule.

When the fluorescent DNA strand separates from the unlabeled strand a second DNA replication reaction can begin. In this reaction the fluorescent single stranded DNA becomes the template DNA. Unlabeled dNTPs, the appropriate buffer, a primer and a DNA polymerase bound to a FRET donor fluorophore are added to the reaction. The primer binds to the target sequence on fluorescent template DNA at the determined annealing temperature. At the appropriate conditions, DNA polymerase can then extend that primer by adding normal, unlabeled, nucleotides. As DNA polymerase copies the fluorescent template strand, it passes over the FRET acceptor fluorophore attached to the nucleotides that makes up the fluorescent template strand (see FIG. 3). A laser excites the donor fluorophore on the DNA polymerase causing a FRET reaction between the donor fluorophore and the acceptor fluorophore. The reaction is then detected and the wavelength of the emitted light is correlated to a specific nucleotide.

Many different donor fluorophore-DNA polymerase fusion proteins may have to be made and screened to find an optimal configuration. For example, you may find that for a particular combination of donor fluorophore and DNA polymerase that the donor fluorophore provides a better FRET reaction when fused on the C terminus instead of the N terminus of the polymerase. U.S. Pat. No. 6,982,146 provides good methods for screening this type of fusion protein.

There are many different clinical applications of this patent. Potentially, entire genomes could be sequenced within days at a relatively inexpensive price. It will offer individuals the potential to sequence their own genomes opening a new frontier into medicine, disease prevention, and research.

Because of the potential for many different embodiments of this novel DNA sequencing method, it should be noted that the illustrated embodiments are only examples of how this method could be performed and should not limit the scope of this patent.

### BRIEF DESCRIPTION OF THE FIGURES

FIG. 1—DNA polymerase synthesizes a fluorescent DNA strand.

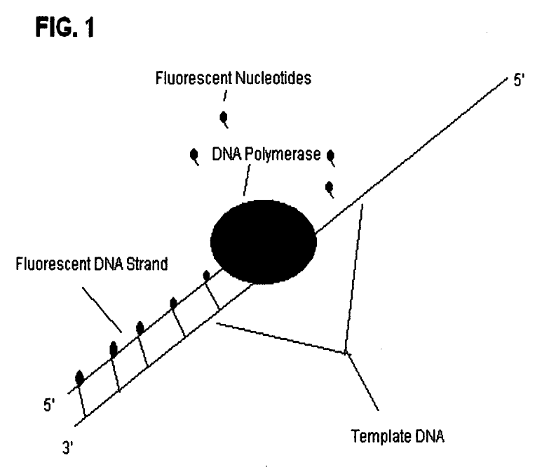

FIG. 2—The double stranded DNA is isolated. A DNA scanning protein complex carrying a donor fluorophore is excited by a laser as it scans the DNA. The donor fluorophore in turn excites the acceptor fluorophore on the fluorescent DNA strand.

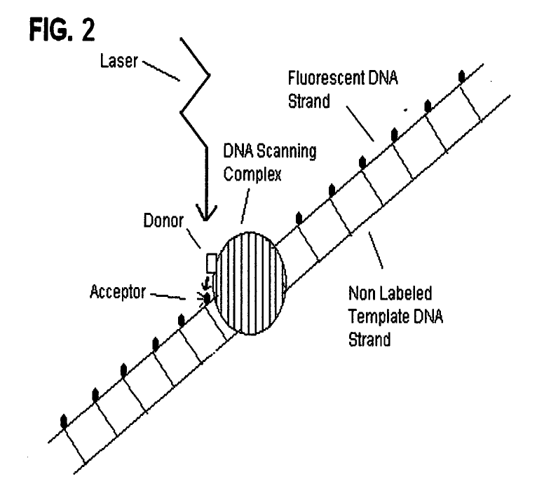

FIG. 3—The fluorescent DNA stand is isolated and used as the new template. A DNA polymerase carrying a donor fluorophore is excited by a laser as it builds the complementary strand. The donor fluorophore in turn excites the acceptor fluorophore on the fluorescent DNA strand.

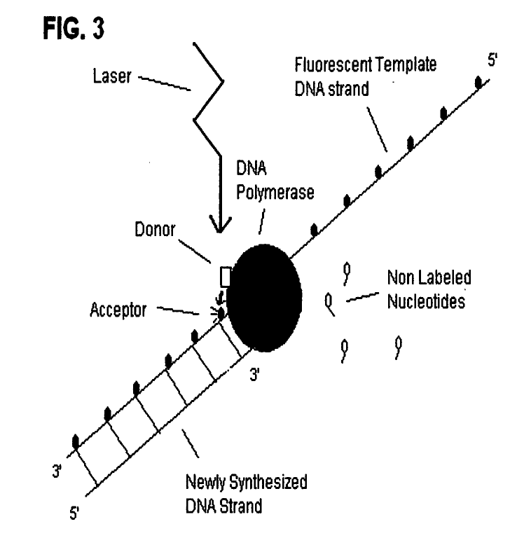

1. A method to Sequence Nucleic Acids, comprising:
A. A fluorescently labeled nucleic acid molecule.
B. A protein, or protein complex, that has the ability to move along a nucleic acid molecule and is attached to at least one fluorescent molecule.
Whereby a FRET reaction can occur between the said fluorescently labeled nucleic acid molecule and the said protein when a fluorescent molecule is excited.

2. The method of claim 1 wherein the said fluorescently labeled nucleic acid molecule is composed of A, T, G, and C and are each bound to a different and distinguishable fluorescent molecule.

3. The method of claim 1 wherein the said fluorescently labeled nucleic acid molecule are composed of A, U, G, and C and are each bound to a different and distinguishable fluorescent molecule.

4. The method of claim 1 wherein the said nucleic acid molecule is double stranded DNA and is fluorescently labeled only on one strand.

5. The method of claim 1 wherein the said nucleic acid molecule is single stranded DNA.

6. The method of claim 1 wherein the said nucleic acid molecule is RNA.

7. The method of claim 1 wherein unincorporated fluorescently labeled nucleotides are removed from the reaction before the addition of said protein.

8. The method of claim 1 wherein the said protein is a protein from the mismatch repair protein complex.

9. The method of claim 1 wherein the said protein is a DNA polymerase which moves along a single stranded of the said fluorescently labeled nucleic acid molecule and adds unlabled, normal, nucleotides.

10. The method of claim 1 wherein the fluorescent molecules on said fluorescently labeled nucleic acid molecule is FRET acceptors and the fluorescent molecule on the said protein is FRET donor molecules.

11. The method of claim 1 wherein the fluorescent molecules on said fluorescently labeled nucleic acid molecule is FRET donors and the fluorescent molecule on the said protein is FRET acceptor molecules.

12. The method of claim 1 wherein a laser specifically excites the fluorescent molecule on the said protein.

13. The method of claim 1 wherein a laser specifically excites the fluorescent molecule on the said fluorescently labeled nucleic acid molecule.

14. The method of claim 1 wherein the said FRET reaction is detected and correlated to a specific nucleotide allowing one to determine the sequence of the said fluorescently labeled nucleic acid molecule.

15. The method of claim 1 where many of the said fluorescently labeled nucleic acid molecules are attached to a solid surface and many of the said FRET reactions occur simultaneously.

16. The method of claim 1 where many of the said proteins are attached to a solid surface and many of the said FRET reactions occur simultaneously.

17. The method of claim 1 where a software program analyses the emission spectra from the said FRET reaction and correlates the said emission spectra to a the appropriate nucleotide.

18. The method of claim 1 wherein the fluorescent molecule on the said protein is a luminescent molecule.

19. The method of claim 1 where a zero-mode waveguide nanostructure arrays are used to detect the said FRET reaction.

20. The method of claim 1 where a microscope is used to detect the said FRET reaction.
